(19) United States
(12) Patent Application Publication
(10) Pub. No.: US 2026/0263346 A1
(43) Pub. Date: Sep. 10, 2026

(54) ALCOHOL-FREE FRAGRANCE COMPOSITION

(71) Applicant: ITC Limited, New Delhi (IN)

(72) Inventors: Pravalika Mohan, Bangalore (IN); Shravya Madnur, Bangalore (IN); Avinash A. Kumar, Bangalore (IN); Vijayan Padmanabhan, Bangalore (IN)

(73) Assignee: ITC Limited, New Delhi (IN)

(21) Appl. No.: 19/163,511

(22) PCT Filed: Dec. 11, 2024
(86) PCT No.: PCT/IB2024/062492

§ 371 Date: Sep. 9, 2025

(30) Foreign Application Priority Data
Dec. 12, 2023 (IN) 202341084827

(51) Int. Cl. A61K 8/92; A61K 8/34; A61K 8/86; A61Q 13/00

(52) U.S. Cl. CPC A61K 8/92; A61K 8/342; A61K 8/86; A61Q 13/00; A61K 2800/30

(57) ABSTRACT

The present disclosure provides an alcohol-free and in particular ethanol-free aqueous fragrance composition, comprising: a solubilizer system comprising at least two non-ionic surfactants, a solvent system, a fragrance oil, and water.

### TECHNICAL FIELD

The present disclosure relates to a fragrance composition. In particular, the present disclosure relates to an alcohol-free fragrance composition, and to consumer products incorporating such fragrance composition.

### BACKGROUND

The background description includes information that may be useful in understanding the present invention. It is not an admission that any of the information provided herein is prior art or relevant to the presently claimed invention, or that any publication specifically or implicitly referenced is prior art.

Fragrance compositions typically contain an alcohol, in particular ethanol, as a solvent because fragrance oils, such as essential oils, dissolve well in alcohol. However, ethanol is a volatile organic compound (VOC) and is subject to control and regulation by the environmental protection agencies. Solid perfumes (e.g., solid emulsions, gels, and pomades) and scented oils are an alternative to alcohol-based fragrance compositions. However, their main disadvantage is that they leave greasy and slippery spots on the perfumed surfaces.

Water-based fragrance compositions have also been reported in the prior art. However, water-based compositions also have numerous drawbacks, including: limitations on the amount of fragrance oil that can be used in the formulation due to the hydrophobic (water-insoluble) nature of fragrance oils; the need of large amounts of solubilizing agents that result in highly viscous or foaming product not suitable for use in pump spray dispensers; the need of high shear forces or complicated ultrasonic techniques to form oil droplets in a nano/micro meter range in order to provide clear, homogeneous nano/micro emulsions; and undesirable skin feel such as tackiness and skin irritation.

Despite the improvements of the water-based fragrance compositions, many problems remain unsolved, or their solutions remain unsatisfactory. There is thus a need in the art for an alcohol-free aqueous fragrance composition that overcomes the aforementioned disadvantages of the prior-art compositions and provides overall satisfactory results. The present disclosure satisfies these needs and provides further related advantages.

### OBJECTS

It is an object of the present disclosure to provide an alcohol-free aqueous fragrance composition.

It is another object of the present disclosure to provide an alcohol-free aqueous fragrance composition that comprises a higher loading of fragrance oil.

It is another object of the present disclosure to provide an alcohol-free aqueous fragrance composition that is storage stable over a wide temperature range.

It is another object of the present disclosure to provide an alcohol-free aqueous fragrance composition that can be formulated without the need of high shear forces or ultrasonic techniques to disperse or dissolve fragrance oils in water phase.

It is another object of the present disclosure to provide an alcohol-free aqueous fragrance composition that is sprayable.

It is another object of the present disclosure to provide an alcohol-free aqueous fragrance composition, which is non-sticky, non-greasy and/or non-irritating.

It is another object of the present disclosure to provide an alcohol-free aqueous fragrance composition that provides enhanced sensorial properties when applied to human skin.

It is another object of the present disclosure to provide an alcohol-free aqueous fragrance composition which can be adapted for a wide range of personal care products.

It is yet another object of the present disclosure to provide a convenient and inexpensive process for producing an alcohol-free aqueous fragrance composition.

### SUMMARY

The above and other objects are met by the present disclosure which, in one aspect, provides an alcohol-free and in particular ethanol-free aqueous fragrance composition, comprising:
a solubilizer system comprising at least two non-ionic surfactants;
a solvent system;
a fragrance oil; and
water.

According to embodiments of the present disclosure, the at least two non-ionic surfactants can be selected from the group consisting of pareth-7, pareth-9, pareth-12, PEG-40-hydrogenated castor oil, sorbitan monolaurate, Laureth-12, Laureth-10, Deceth-12, Cetearth-20, Ceteareth-12, Laureth-23, Pareth-20, Polyglyceryl-4 caprate, PEG-40 stearate, and Laureth-2.

In some embodiments, the solubilizer system comprises two non-ionic surfactants. The two non-ionic surfactants belong to different classes of non-ionic surfactants, each class being selected from the group consisting of PEG alkyl ether, PEGylated solubilizers, and ethoxylated alcohols.

In some embodiments, the solubilizer system comprises two non-ionic surfactants, which are Pareth-12 and PEG-40 hydrogenated castor oil. In some embodiments, the solubilizer system comprises two non-ionic surfactants, which are Laureth-10 and PEG-40 hydrogenated castor oil. In some embodiments, the amount of Pareth-12 in the fragrance composition can range from 1.5 wt. % to 10 wt. % based on the total weight of the fragrance composition. In some embodiments, the amount of Laureth-10 in the fragrance composition can range from 1.5 wt. % to 10 wt. % based on the total weight of the fragrance composition. In some embodiments, the amount of PEG-40 hydrogenated castor oil in the fragrance composition can range from 1 wt. % to 10 wt. % based on the total weight of the fragrance composition. In some embodiments, the weight ratio of PEG-40 hydrogenated castor oil to Pareth-12/Laureth-10 in the fragrance composition ranges from 1:3 to 3:1.

The solvent system comprises at least one polyglycol ether selected from the group consisting of diethylene glycol monoethyl ether (DEGMEE), ethylene glycol monoethyl ether, diethylene glycol monobutyl ether, triethylene glycol monoethyl ether, ethylene glycol monobutyl ether, and dipropylene glycol monoethyl ether. The amount of the polyglycol ether in the fragrance composition ranges from 0.2 wt. % to 2.6 wt. % based on the total weight of the fragrance composition.

The solvent system may further include one or more polyols selected from the group consisting of dipropylene glycol, 1,3-propanediol, butylene glycol, hexylene glycol, and 2-methyl-1,3-propanediol. The amount of said one or more polyols may range from 0.002 wt. % to 10 wt. % based on the total weight of the fragrance composition.

In some of the exemplified embodiments, the solvent system comprises a combination of DEGMEE and dipropylene glycol.

The fragrance oil can be used in amounts ranging from 3 wt. % to 12 wt. %, based on the total weight of the fragrance composition.

The amount of water used in the alcohol-free aqueous fragrance composition ranges between 60 wt. % and 90 wt. %, preferably between 60 wt. % and 80 wt. %, based on the total weight of the fragrance composition.

In various embodiments, the alcohol-free aqueous fragrance composition is sprayable, e.g., by means of a hand-actuated spray pump dispenser including those especially suitable for atomizing water-based perfumes.

In another aspect, the present disclosure is directed to a process for preparing an alcohol-free, in particular ethanol-free, aqueous fragrance composition. The process includes the steps of:
a. preparing a phase-1 by combining water and optionally a buffering agent to form a homogenous aqueous phase;
b. preparing a phase-2 by combining a solubilizer system, a solvent system, a fragrance oil, and optionally a preservative and a UV stabilizer;
c. adding the phase-2 to the phase-1, and homogenizing the combined phases; and
d. optionally mixing an anti-foaming agent with the combined phases to obtain a stable, clear aqueous fragrance composition.

Various objects, features, aspects and advantages of the inventive subject matter will become more apparent from the following detailed description of preferred embodiments.

### DETAILED DESCRIPTION

The following is a detailed description of embodiments of the present disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Alcohol in this context refers to ethyl alcohol. Near zero means <0.03% by weight of the formulation.

A formulation is considered to be stable if it does not exhibit any phase separation or turbidity (NTU value≤30) at room temperature when stored over a period of 2 weeks to 36 months. It must also not exhibit any phase separation and/or turbidity (NTU value≤30) when stored at lower temperatures (4 to 10° C.) or higher temperatures (45-55° C.) for a continuous period of 24-36 hours.

The term “fragrance oil” as used herein is to be construed broadly to include natural and/or synthetic aromatic oils and blends thereof wherein at least 70 wt. % of the constituents are characterized by log P value below 2.

The terms “fragrance oil”, “fragrance component”, “perfume oil” and “perfuming ingredient” are interchangeable terms having the same meaning throughout the specification.

The present disclosure provides an alcohol-free and in particular ethanol-free aqueous fragrance composition, comprising:
a solubilizer system comprising at least two non-ionic surfactants;
a solvent system;
a fragrance oil; and
water.

As indicated above, the fragrance composition of the present disclosure employs a solubilizer system comprising at least two non-ionic surfactants in combination with a solvent system to disperse or dissolve the fragrance oil in water. Surprisingly, it has been found that the specific combination of the non-ionic surfactants with the solvent system produces an unexpected solubilization effect which increases the solubility of fragrance oil(s) in water, thereby facilitating higher loading of fragrance oils into the aqueous phase and their uniform distribution, without any counterproductive effects on stability, viscosity and sensorial properties of the fragrance composition, and which allows for direct transfer of fragrance oil(s) into water to form a single-phase, clear and temperature stable fragrance composition, without the need to apply high agitation forces, such as high shear or ultrasonic mixing.

According to embodiments of the present disclosure, the at least two non-ionic surfactants are selected from the group consisting of pareth-7, pareth-9, pareth-12, PEG-40-hydrogenated castor oil, sorbitan monolaurate, Laureth-12, Laureth-10, Deceth-12, Cetearth-20, Ceteareth-12, Laureth-23, Pareth-20, Polyglyceryl-4 caprate, PEG-40 stearate, and Laureth-2. The at least two non-ionic surfactants are present in the fragrance composition in a total amount of from 2 wt. % to 20 wt. % based on the total weight of the fragrance composition.

In some embodiments, the solubilizer system comprises two non-ionic surfactants. The two non-ionic surfactants belong to different classes of non-ionic surfactants, each class being selected from the group consisting of PEG alkyl ether, PEGylated solubilizers, and ethoxylated alcohols. In an embodiment, the two non-ionic surfactants are selected from the group consisting of pareth-7, pareth-9, pareth-12, PEG-40-hydrogenated castor oil, sorbitan monolaurate, Laureth-12, Laureth-10, Deceth-12, Cetearth-20, Ceteareth-12, Laureth-23, Pareth-20, Polyglyceryl-4 caprate, PEG-40 stearate, and Laureth-2. A combined weight percentage of the two non-ionic surfactants ranges from 2.5 wt. % to 20 wt. % based on the total weight of the fragrance composition.

In some embodiments, the solubilizer system comprises two non-ionic surfactants, which are Pareth-12 and PEG-40 hydrogenated castor oil. In some embodiments, the solubilizer system comprises two non-ionic surfactants, which are Laureth-10 and PEG-40 hydrogenated castor oil. In some embodiments, the amount of Pareth-12 in the fragrance composition can range from 1.5 wt. % to 10 wt. % based on the total weight of the fragrance composition. In some embodiments, the amount of Laureth-10 in the fragrance composition can range from 1.5 wt. % to 10 wt. % based on the total weight of the fragrance composition. In some embodiments, the amount of PEG-40 hydrogenated castor oil in the fragrance composition can range from 1 wt. % to 10 wt. % based on the total weight of the fragrance composition. In some embodiments, the weight ratio of PEG-40 hydrogenated castor oil to Pareth-12/Laureth-10 in the fragrance composition ranges from 1:4 to 4:1, preferably 1:3 to 3:1.

The solvent system comprises at least one polyglycol ether selected from the group consisting of diethylene glycol monoethyl ether (DEGMEE), ethylene glycol monoethyl ether, diethylene glycol monobutyl ether, triethylene glycol monoethyl ether, ethylene glycol monobutyl ether, and dipropylene glycol monoethyl ether. The amount of the polyglycol ether in the fragrance composition ranges from 0.2 wt. % to 2.6 wt. % based on the total weight of the fragrance composition.

The solvent system may further include one or more polyols selected from the group consisting of dipropylene glycol, 1,3-propanediol, butylene glycol, hexylene glycol, and 2-methyl-1,3-propanediol. The amount of said one or more polyols may range from 0.002 wt. % to 10 wt. % based on the total weight of the fragrance composition.

In certain embodiments, the solvent system comprises a combination of DEGMEE and dipropylene glycol.

Fragrance oil is a mixture of aromatic compounds whose primary function is to create fragrance or modulate malodor. As used herein, the term “fragrance oil” maintains its typical and usual meaning in the art, i.e. a lipophilic organic liquid that is essentially insoluble in water and being composed of one or more ingredients of natural and/or synthetic source that produce any kind of olfactive response including attractive, pleasant, flowery, aromatic, pungent, and woody odors. Particularly, the fragrance oil includes any perfuming ingredient or any mixture of perfuming ingredients currently used in perfumery, e.g. compounds capable of imparting a hedonic olfactive effect to the composition to which they are added. The perfuming ingredients can be of natural or synthetic origin. In general, the perfuming ingredients belong to a varied class of compounds including, but not limited to, alcohols (other than ethanol), ketones, aldehydes, terpenoids, ethers, esters, acetates, and essential oils of natural or synthetic origin. The term “fragrance oil” as used herein is to be construed broadly to include commercially available fragrance oils wherein at least 70 wt. % of the constituents are characterized by log P value below 2.

Examples of fragrance oils that can be used in the composition of the present disclosure include: C6-C12 aldehydes, anisaldehyde, acetal R, acetophenone, acetylcedrene, adoxal, allylamyl glycolate, allyl cyclohexanepropionate, a-damascone, β-damascone, δ-damascone, ambrettolide, ambroxan, amylcinnamic aldehyde, amylcinnamic aldehyde dimethylacetal, amyl valerianate, amyl salicylate, isoamyl acetate, isoamyl salicylate, aurantiol, acetyl eugenol, bacdanol, benzyl acetate, benzyl alcohol, benzyl salicylate, bergamyl acetate, bornyl acetate, butyl butyrate, p-t-butylcyclohexanol, p-t-butylcyclohexyl acetate, o-t-butylcyclohexanol, benzaldehyde, benzyl formate, caryophyllene, cashmerane, carvone, cedramber, cedryl acetate, cedrol, celestolide, cinnamic alcohol, cinnamic aldehyde, cis-jasmone, citral, citral dimethyl acetal, citrasal, citronellal, citronellol, citronellyl acetate, citronellyl formate, citronellyl nitrile, cyclaset, cyclamen aldehyde, cyclaprop, caron, coumarin, cinnamyl acetate, 6-C6-C13 lactone, dimethylbenzylcarbinol, dihydrojasmon, dihydrolinalool, dihydromyrcenol, dimetol, dimyrcetol, diphenyl oxide, ethyl vanillin, eugenol, fruitate, fenchyl alcohol, phenylethyl phenyl acetate, galaxolide, γ-C6-C13 lactone, geraniol, geranyl acetate, geranyl formate, geranyl nitrile, hedion, helional, heliotropin, cis-3-hexanol, cis-3-hexenyl acetate, cis-3-hexenyl salicylate, hexylcinnamic aldehyde, hexyl salicylate, hyacinth dimethyl acetal, hydrotropic alcohol, hydroxycitronellal, indole, ionone, isobornyl acetate, isocyclocitral, Iso E Super, isoeugenol, isononyl acetate, isobutylquinoline, jasmal, jamolactone, jasmopirane, corvone, ligustoral, lilial, lime oxide, limonene, linalool, linalool oxide, linalyl acetate, lyral, manzanate, myol, menthanyl acetate, menthonate, methyl anthranilate, methyl eugenol, menthol, a-methylionone, β-methylionone, γ-methylionone, methyl isoeugenol, methyl lavender ketone, methyl salicylate, muguet aldehyde, mugol, musk TM-II, musk 781, musk C14, musk T, musk ketone, musk tibetene, musk moskene, myrac aldehyde, methylphenyl acetate, nerol, neryl acetate, nopyl acetate, nopyl alcohol, neobergamate, oak moss No. 1, orivone, oxyphenylon, p-cresyl methyl ether, pentalide, phenylethyl alcohol, phenylethyl acetate, phenylacetaldehyde, dimethyl acetal, a-pinene, rubafuran, rosephenone, rose oxide, Sandalore, Sandela, Santalex, Santalinol, styralyl acetate, styralyl propionate, terpineol, terpinyl acetate, tetrahydrolinalool, tetrahydrolinalyl acetate, tetrahydrogeraniol, tetrahydrogeranyl acetate, tonalide, traseolide, tripral, thymol, vanillin, verdox, yara yara, anis oil, bay oil, bois de-rose oil, cananga oil, cardamon oil, cassia oil, cedarwood oil, orange oil, mandarin oil, tangerine oil, basil oil, nutmeg oil, citronella oil, clove oil, coriander oil, elemi oil, eucaryptus oil, fennel oil, galbanum oil, geranium oil, hiba oil, hinoki oil, jasmine oil, lavandin oil, lavender oil, lemon oil, lemongrass oil, lime oil, neroli oil, oak moss oil, ocotea oil, patchouli oil, peppermint oil, perilla oil, petitgrain oil, pine oil, rose roil, rosemary oil, camphor oil, ho leaf oil, clary sage oil, sandalwood oil, spearmint oil, spike lavender oil, star anise oil, thyme oil, tonka bean tincture, turpentine oil, vanilla bean tincture, vetiver oil, bergamot oil, ylang ylang oil, grapefruit oil, yuzu (Citrus Junos Tanaka) oil, benzoin, balsam peru, balsam tolu, tuberose oil, musk tincture, castrium tincture, civet tincture, ambergris tincture, and mixtures thereof.

The terms “fragrance oil”, “fragrance component”, “perfume oil” and “perfuming ingredient” are interchangeable terms having the same meaning throughout the specification.

In various embodiments, the fragrance oil may be used in the alcohol-free aqueous fragrance composition in amounts between 3 wt. % and 12 wt. %, based on the total weight of the fragrance composition.

In some embodiments, the ratio of the fragrance oil to the solvent system in the composition is lower than 1. In some embodiments, the ratio of the fragrance oil to the solvent system in the composition is higher than 1.

The amount of water used in the alcohol-free aqueous fragrance composition ranges between 60 wt. % and 90 wt. %, preferably between 60 wt. % and 80 wt. %, based on the total weight of the fragrance composition.

In an embodiment, the overall irritancy score of the alcohol-free aqueous fragrance composition as measured by Colorimetric Index of Mildness through corneosurfametry is above 30 which is considered to be non-irritant and mild on skin.

In some embodiments, the alcohol-free aqueous fragrance composition as described herein can further include an antifoaming agent, such as simethicone. When present, the amount of such antifoaming agent is between 0.001 wt. % and 0.03 wt. %, based on the total weight of the fragrance composition.

In various embodiments, the alcohol-free aqueous fragrance composition as described herein can further include one or more cosmetic ingredients such as emollients, humectants, cooling agents, vitamins, antioxidants, buffering agents, preservatives and/or UV stabilizers. These ingredients are well known in the field of perfumery and the proportions are established and known to those skilled in the art. For example, said cosmetic/perfumery ingredients are described in “Perfume and Flavour Chemicals”, S. Arctander, Allured Publishing Corporation, 1994, IL, USA, which is incorporated herein by reference.

In various embodiments, the specific combination of the solubilizer system and the solvent system according to the present disclosure enables to prepare a transparent sprayable fragrance composition that exhibits stability at room temperature for a period ranging from 2 weeks to 36 months.

In various embodiments, the alcohol-free aqueous fragrance composition as described herein remains stable for a period of 3 months to 9 months at a temperature ranging from 5° C. to 50° C.

In various embodiments, the alcohol-free aqueous fragrance composition as described herein remains clear (turbidity less than or equal to 30 NTU) and stable over at least 24 hours of continuous exposure to temperatures as low as 5° C. and as high as 50° C.

In various embodiments, the alcohol-free aqueous fragrance composition is sprayable, e.g., by means of a hand-actuated spray pump dispenser including those especially suitable for atomizing water-based perfumes.

In various embodiments, the alcohol-free aqueous fragrance composition has a viscosity in the range of from about 5 cps to about 15 cps.

The alcohol-free aqueous fragrance composition as described herein is in the form of a leave-on composition. As used herein, the term “leave-on composition” refers to compositions that are applied on a surface, e.g., human skin, without a subsequent step of washing the surface to remove the composition after the application. This implies that the compositions stays on the surface after the application.

In various embodiments, the alcohol-free aqueous fragrance composition as described herein can be formulated into personal care products or home care products.

In some embodiments, the alcohol-free aqueous fragrance composition is formulated into personal care preparations for leave-on application to skin, such as, perfume spray, deodorant spray, and face/body sprays.

In some embodiments, the alcohol-free aqueous fragrance composition as described herein comprises a fragrance oil suitable for air freshening purposes.

In another aspect, the present disclosure is directed to a process for preparing an alcohol-free, in particular ethanol-free, aqueous fragrance composition. The process includes the steps of:
a. preparing a phase-1 by combining water and optionally a buffering agent to form a homogenous aqueous phase;
b. preparing a phase-2 by combining a solubilizer system, a solvent system, a fragrance oil, and optionally a preservative and a UV stabilizer;
c. adding the phase-2 to the phase-1, and homogenizing the combined phases; and
d. optionally mixing an anti-foaming agent with the combined phases to obtain a stable clear aqueous fragrance composition.

All components and features for the process are as defined above for the fragrance composition.

In one exemplary embodiment, the solubilizer system comprises a PEGylated solubilizer such as PEG-40 hydrogenated castor oil in combination with a PEG alkyl ether such as C12-15 Pareth-7 to 12.

In another exemplary embodiment, the solubilizer system comprises a PEGylated solubilizer such as PEG-40 hydrogenated castor oil in combination with a PEG alkyl ether such as C12-14 Laureth-7 to 15.

In a further exemplary embodiment, the solvent system comprises a combination of a polyglycol ether, e.g., DEGMEE and a polyol, e.g., dipropylene glycol.

The alcohol-free aqueous fragrance composition of the present disclosure shows much higher retention of fragrance ingredients on a substrate compared to alcohol-based formulations. In some embodiments, the alcohol-free aqueous fragrance composition shows retention of some of the desirable fragrance ingredients on a substrate that is at least 30% higher than the amount otherwise retained in case of alcohol-based formulations.

In some embodiments, the alcohol-free aqueous fragrance composition of the present disclosure is substantially free of silicone carriers. These near-zero silicone (less than 0.03 wt. %) formulations may only carry small quantities of silicone-based antifoaming agents such as simethicone.

While the foregoing description discloses various embodiments of the disclosure, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### EXAMPLES

The present disclosure is further explained in the form of following examples. However, it is to be understood that the following examples are merely illustrative and are not to be taken as limitations upon the scope of the invention. Various changes and modifications to the disclosed embodiments will be apparent to those skilled in the art. Such changes and modifications may be made without departing from the scope of the invention. The appropriate procedure to prepare the fragrance compositions will be apparent to those skilled in the art.

### Working Examples

Examples 1 to 8 were prepared according to the ingredients and amounts shown in Table 1 below. Examples 1 to 8 were found to be stable without any phase separation or turbidity (NTU value≤30) at room temperature when stored over a period of 2 weeks to 36 months, further they also did not exhibit any phase separation and/or turbidity (NTU value≤30) when stored at lower temperatures (4 to 10° C.) or higher temperatures (45-55° C.) for a continuous period of 24-36 hours.

TABLE 1

| | (% w/w) | | | | | | | |
|---|---|---|---|---|---|---|---|---|
| Ingredient | Ex 1 | Ex 2 | Ex 3 | Ex 4 | Ex 5 | Ex 6 | Ex 7 | Ex 8 |
| C12-14 Laureth-10/ | 4 | 5 | 5 | 4 | 7 | 8 | 3 | 5 |
| C12-C15 Pareth-12 | | | | | | | | |
| PEG-40 hydrogenated | 3 | 5 | 5 | 5 | 3 | 8 | 7 | 5 |
| castor oil | | | | | | | | |
| Dipropylene glycol | 4 | 5 | 5 | 5 | 5 | 7.5 | 5 | 5.4 |
| DEGMEE | 2 | 2.5 | 2.5 | 2.5 | 2.5 | 2.5 | 2.5 | 1.8 |
| Fragrance oil* | 6 | 6 | 6 | 7 | 6 | 10 | 6 | 6 |
| Tert-butyl alcohol | 0.9 | — | 0.9 | 0.9 | 0.9 | 0.9 | 0.9 | 0.9 |
| Excipients | Q.S | Q.S | Q.S | Q.S | Q.S | Q.S | Q.S | Q.S |
| Water | Q.S. | Q.S. | Q.S. | Q.S. | Q.S. | Q.S. | Q.S. | Q.S. |
| | to 100 | to 100 | to 100 | to 100 | to 100 | to 100 | to 100 | to 100 |
| Observation | Clear at | Clear at | Clear at | Clear at | Clear at | Clear at | Clear at | Clear at |
| | RT* and at | RT and at | RT and at | RT and at | RT and at | RT and at | RT and at | RT and at |
| | elevated | elevated | elevated | elevated | elevated | elevated | elevated | elevated |
| | and lower | and lower | and lower | and lower | and lower | and lower | and lower | and lower |
| | temperatures | temperatures | temperatures | temperatures | temperatures | temperatures | temperatures | temperatures |

RT refers to room temperature.
Elevated temperature refers to temperature ranging from about 45° C. to about 55° C.
Lower temperature refers to temperature ranging from about 4° C. to about 10° C.
Measurements and observations are taken within 15 minutes of removal from respective conditions.
Excipients include one or more of emollients, humectants, cooling agents, vitamins, antioxidants, buffering agents, preservatives and/or UV stabilizers and antifoaming agents.
In Ex 1 to 4, the fragrance oil used was Mesmerize LF Mod Max 2 from IFF. In Ex 5 to 8, the fragrance oil used was Frozen Throne from Robertet.

### COMPARATIVE EXAMPLES

Comparative Examples 1 to 10 were prepared according to the ingredients and amounts shown in Table 2 below:

TABLE 2

| | (% w/w) | | | | | | | | | |
|---|---|---|---|---|---|---|---|---|---|---|
| | C. | C. | C. | C. | C. | C. | C. | C. | C. | C. |
| Ingredient | Ex 1 | Ex 2 | Ex 3 | Ex 4 | Ex 5 | Ex 6 | Ex 7 | Ex 8 | Ex 9 | Ex 10 |
| PEG-40 | 7 | — | 1 | 1 | 3 | — | 0.9 | 5 | 5 | 3 |
| hydrogenated | | | | | | | | | | |
| castor oil | | | | | | | | | | |
| Decyl | | | | | 4 | | | | | |
| glucoside | | | | | | | | | | |
| Polysorbate- | | | | | | | | | | 4 |
| 20 | | | | | | | | | | |
| Propylene | | | | | | | | 5 | 2.5 | |
| glycol | | | | | | | | | | |
| C12-15 | — | 7 | 7 | 7 | — | 7 | 1.4 | 5 | 5 | |
| Pareth-12 | | | | | | | | | | |
| Dipropylene | 4 | 4 | 2 | 4 | 4 | — | 0.001 | — | 5 | 4 |
| glycol | | | | | | | | | | |
| DEGMEE | 2 | 2 | 2 | — | 2 | — | 0.1 | 2.5 | — | 2 |
| Fragrance oil | 6 | 6 | 6 | 6 | 6 | 6 | 3.5 | 6 | 6 | 6 |
| Tert-butyl | 0.9 | 0.9 | 0.9 | 0.9 | 0.9 | 0.9 | 0.9 | 0.9 | 0.9 | 0.9 |
| alcohol | | | | | | | | | | |
| Excipients | Q.S | Q.S | Q.S | Q.S | Q.S | Q.S | Q.S | Q.S | Q.S | Q.S |
| Water | Q.S | Q.S | Q.S | Q.S | Q.S | Q.S | Q.S | Q.S | Q.S | Q.S |
| | to 100 | to 100 | to 100 | to 100 | to 100 | to 100 | to 100 | to 100 | to 100 | to 100 |
| Observation | Separation | Separation | Separation | Separation | Hazy at | Separation | Separation | Hazy at | Hazy at | Separation |
| | at RT | at RT | at RT | at RT | RT and | at RT | at RT, | lower | lower | at RT |
| | and lower | and lower | and lower | and lower | lower | and lower | and lower | temper- | temper- | and lower |
| | temper- | temper- | temper- | temper- | temper- | temper- | and | atures | atures | and |
| | atures | atures | atures | atures | atures | atures | higher | | | Higher |
| | and cloudy | | | | and cloudy | | temper- | | | temper- |
| | at higher | | | | at elevate | | atures | | | atures |
| | temper- | | | | temper- | | | | | |
| | atures | | | | atures | | | | | |

RT refers to room temperature
Elevated temperature refers to temperature ranging from about 45° C. to about 55° C.
Lower temperature refers to temperature ranging from about 4° C. to 8° C.
Measurements and observations are taken within 15 minutes of removal from respective conditions
Excipients include one or more of emollients, humectants, cooling agents, vitamins, antioxidants, buffering agents, preservatives and/or UV stabilizers, and antifoaming agents, that are well known to a person skilled in the art.
In C. Ex 1 to C. Ex 5, the fragrance oil used was Mesmerize LF Mod Max 2 from IFF.
In C. Ex 6 to C. Ex 10, the fragrance oil used was Frozen Throne from Robertet.
QS refers to quantity sufficient
QS to 100 means quantity sufficient to make it up to 100.

Comparative examples C. Ex 1 and C. Ex 2 demonstrate that when only PEG-40 hydrogenated castor oil or pareth-12 is used as the solubilizer (non-ionic surfactant), the resulting composition is not stable. Haziness and oil separation were observed in compositions containing only PEG-40 hydrogenated castor oil or pareth-12 as the solubilizer when stored at RT and elevated temperatures, while those containing both PEG-40 hydrogenated castor oil and Laureth-10/Pareth-12 were found to be stable under same storage conditions (see, working examples Ex 1 to Ex 8). This indicated that presence of at least two non-ionic surfactants is necessary to attain the inventive compositions. Further, the inventors during their experimentation also came to a conclusion that the two non-ionics deployed have to be from two different categories. Both in Ex1 and Ex2, the two non-ionic surfactants present belong to two different categories. Without bound by theory, it seems that this heterogeneous mix of non-ionic surfactant seems to have facilitated to attain the desired level of solubilization.

Comparative Ex 5 containing PEG-40 hydrogenated castor oil and decyl glycoside as the non-ionic surfactants and C. Ex 10 containing PEG-40 Hydrogenated Castor Oil and Polysorbate-20 failed to provide a stable product, while those containing the specific combination of non-ionic surfactants according to the present disclosure demonstrated desired stability. The inventors have observed that the choice of categories of non-ionic surfactant could also in a way dictate not only fragrance solubility but also the fragrance loading capacity and the aesthetics altogether. Apart from decyl glucoside, an alkyl glycoside, other classes of non-ionic surfactants, for example polysorbates outside the inventive scope also did not work. This demonstrates that the choice of non-ionic surfactants is critical to obtaining a stable product.

Comparative examples C. Ex 3 and C. Ex 4 demonstrate that when the non-ionic surfactants are used in a ratio outside the range of the present disclosure, the resulting composition is not stable.

When the non-ionic surfactants were used without a solvent system, as in the comparative example C. Ex 6, the resulting compositions were found to exhibit instability. The solvent system very much like the solubilizers essentially aids in stability, aesthetics and sensorial.

The composition of comparative example C. Ex 7 demonstrates that when the non-ionic surfactants are used in a concentration outside the range of the present disclosure, the resulting composition is not stable.

The compositions of comparative examples C. Ex 8 and C. Ex 9 not containing either dipropylene glycol or DEGMEE, but instead containing propylene glycol as the solvent, became hazy at elevated temperatures during storage, while those containing the specific solvents (solvent system) according to the present disclosure were stable at the same conditions (see, working examples Ex 1 to Ex 8). This demonstrates that the choice of solvent is critical to the stability as well as aesthetics of the composition.

Examples wherein the polyglycol ethers (DEGMEE) are used beyond 2.5% were not considered, as beyond 2.6% there are regulatory restrictions on the use of DEGMEE. On the other hand, examples with higher proportions (in excess of 10 wt. %) of dipropylene glycol have not been included since these would go against the object of this invention, that is, to use least possible amounts of polyols.

We claim:

1. An alcohol-free aqueous fragrance composition, comprising:
a solubilizer system;
a solvent system;
a fragrance oil; and
water.

2. The fragrance composition as claimed in claim 1, wherein the composition is ethanol-free.

3. The fragrance composition as claimed in claim 1, wherein the solubilizer system comprises two non-ionic surfactants.

4. The fragrance composition as claimed in claim 3, wherein the non-ionic surfactants are selected from the group consisting of pareth-7, pareth-9, pareth-12, PEG-40-hydrogenated castor oil, sorbitan monolaurate, Laureth-12, Laureth-10, Deceth-12, Cetearth-20, Ceteareth-12, Laureth-23, Pareth-20, Polyglyceryl-4 caprate, PEG-40 stearate, and Laureth-2.

5. The fragrance composition as claimed in claim 4, wherein a combined weight percentage of the two non-ionic surfactants ranges from 2.5 wt. % to 20 wt. % based on the total weight of the fragrance composition.

6. The fragrance composition as claimed in claim 1, wherein the solubilizer system comprises Pareth-12 and PEG-40 hydrogenated castor oil.

7. The fragrance composition as claimed in claim 1, wherein the solubilizer system comprises Laureth-10 and PEG-40 hydrogenated castor oil.

8. The fragrance composition as claimed in claim 1, wherein the solvent system comprises at least one polyglycol ether selected from the group consisting of diethylene glycol monoethyl ether (DEGMEE), ethylene glycol monoethyl ether, diethylene glycol monobutyl ether, triethylene glycol monoethyl ether, ethylene glycol monobutyl ether, and dipropylene glycol monoethyl ether.

9. The fragrance composition as claimed in claim 1, wherein the solvent system is present in the fragrance composition in an amount ranging from 0.2 wt. % to 2.6 wt. % based on the total weight of the fragrance composition.

10. The fragrance composition as claimed in claim 1, wherein the solvent system further comprises one or more polyols selected from the group consisting of dipropylene glycol, 1,3-propanediol, butylene glycol, hexylene glycol, and 2-methyl-1,3-propanediol.

11. The fragrance composition as claimed in claim 1, wherein the solvent system comprises a combination of DEGMEE and dipropylene glycol.

12. The fragrance composition as claimed in claim 1, wherein the fragrance oil is present in the fragrance composition in an amount ranging from 3 wt. % to 12 wt. % based on the total weight of the fragrance composition.

13. The fragrance composition as claimed in claim 1, wherein the water is present in the fragrance composition in an amount ranging from 60 wt. % to 90 wt. % based on the total weight of the fragrance composition.

14. The fragrance composition as claimed in claim 1, further comprising one or more cosmetic ingredients selected from the group consisting of an antifoaming agent, an emollient, a humectant, a cooling agent, a vitamin, an antioxidant, a buffering agent, a preservative, and a UV stabilizer.

15. A process for preparing an alcohol-free aqueous fragrance composition, comprising the steps of:
a. preparing a phase-1 by combining water and optionally a buffering agent to form a homogenous aqueous phase;
b. preparing a phase-2 by combining a solubilizer system, a solvent system, a fragrance oil, and optionally a preservative and a UV stabilizer;
c. adding the phase-2 to the phase-1, and homogenizing the combined phases; and
d. optionally mixing an anti-foaming agent with the combined phases to obtain a stable clear aqueous fragrance composition.
